(19) United States
(12) Patent Application Publication
(10) Pub. No.: US 2022/0275216 A1
(43) Pub. Date: Sep. 1, 2022

(54) COATED EFFECT PIGMENTS AND PRODUCTION THEREOF

(71) Applicant: Henkel AG & Co. KGaA, Duesseldorf (DE)

(72) Inventors: Torsten LECHNER, Langenfeld (DE); Gabriele WESER, Neuss (DE); Caroline KRIENER, Duesseldorf (DE); Claudia KOLONKO, Remscheid (DE); Ulrike SCHUMACHER, Duesseldorf (DE); Marc NOWOTTNY, Monchengladbach (DE); Juergen SCHOEPGENS, Schwalmtal (DE); Phillip JAISER, Langenfeld (DE); Carsten MATHIASZYK, Essen (DE); Jing HODES, Hagen (DE)

(73) Assignee: Henkel AG & Co. KGaA, Duesseldorf (DE)

(21) Appl. No.: 17/628,544

(22) PCT Filed: Jul. 6, 2020
(86) PCT No.: PCT/EP2020/068957

§ 371 Date: Jan. 19, 2022

(30) Foreign Application Priority Data
Jul. 19, 2019 (DE) 10 2019 210 694.3

(51) Int. Cl. C09C 1/00

(52) U.S. Cl. CPC C09C 1/0021; C09C 2200/1054; C09C 2220/106

(57) ABSTRACT

An effect pigment and process of making an effect pigment are provided. An exemplary effect pigment includes a substrate platelet and a coating. The coating includes at least one layer which has been wet-chemically prepared using a metal alkoxide and an organosilicon compound having a basic group.

### CROSS-REFERENCE TO RELATED APPLICATION

This application is a U.S. National-Stage entry under 35 U.S.C. § 371 based on International Application No. PCT/EP2020/068957, filed Jul. 6, 2020, which was published under PCT Article 21(2) and which claims priority to German Application No. 102019210694.3, filed Jul. 19, 2019, which are all hereby incorporated in their entirety by reference.

### TECHNICAL FIELD

The present application relates to effect pigments comprising a substrate platelet and a coating, the coating having at least one layer comprising a metal oxide and/or metal oxide hydrate. The application further describes a process to produce the effect pigments.

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

### BACKGROUND

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed without residue by a few washes with surfactant-comprising cleaning agents. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when the combination of a pigment, an organic silicon compound, a film-forming polymer and a solvent is used on hair, it is possible to create colorations that are particularly resistant to shampooing.

Metallic luster pigments or metallic effect pigments are widely used in many fields of technology. They are used, for example, to color coatings, printing inks, inks, plastics, glasses, ceramic products and preparations for decorative cosmetics such as nail polish. They are exemplified above all by their attractive angle-dependent color impression (goniochromism) and their metallic-looking luster.

Hair with a metallic finish or metallic highlights are in trend. The metallic tone makes the hair look thicker and shinier.

### BRIEF SUMMARY

An effect pigment is provided and includes a substrate platelet and a coating. The coating includes at least one layer which has been wet-chemically prepared using a metal alkoxide and an organosilicon compound having a basic group.

A process of making an effect pigment including a substrate platelet and a coating is also provided. The process includes suspending the substrate platelet in an organic or aqueous solvent. The process further includes coating the substrate platelet by a sol-gel process using a metal alkoxide and an organosilicon compound having a basic group.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

There is a need to provide effect pigments, especially for hair dyeing, which on the one hand have high wash and rub fastness and on the other hand do not negatively affect hair properties such as manageability and feel. For this purpose, it would be desirable if the effect pigments used had a high covering power and could be applied to the hair in thin layers.

The effect pigments should be particularly suitable for dyeing systems that do not require the use of oxidizing agents and/or oxidation dye precursors.

Surprisingly, it has now been found that the tasks can be excellently solved by an effect pigment comprising a) a substrate platelet and b) a coating, wherein the coating comprises at least one layer which has been wet-chemically prepared using a metal alkoxide and an organosilicon compound having a basic group.

It has been shown that hair dyeing's with such effect pigments have a high wash and rub fastness.

The effect pigment has a substrate platelet.

The substrate wafer preferably has an average thickness of at most 150 nm, preferably less than 50 nm, more preferably less than 30 nm, particularly preferably at most 25 nm, for example at most 20 nm. The average thickness of the substrate platelets is at least 1 nm, preferably at least 2.5 nm, particularly preferably at least 5 nm, for example at least 10 nm. Preferred ranges for substrate wafer thickness are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm, and 10 to 20 nm. Preferably, each substrate plate has a thickness that is as uniform as possible.

The substrate plate is preferably monolithic. Monolithic in this context means comprising a single self-contained unit without fractures, stratifications or inclusions, although microstructural changes may occur within the substrate platelet. The substrate platelet is preferably homogeneous in structure, i.e., no concentration gradient occurs within the platelet. In particular, the substrate platelet is not layered and does not have particles or particulates distributed therein.

The size of the substrate platelet can be tailored to the specific application, for example the desired effect on a keratinous material. Typically, the substrate platelets have an average largest diameter of about 2 to 200 μm, especially about 5 to 100 μm.

In a preferred embodiment, the shape factor (aspect ratio), expressed by the ratio of the average size to the average thickness, is at least 80, preferably at least 200, more preferably at least 500, particularly preferably more than 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for 3 minutes.

The substrate platelet can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-) precious stones, and plastics. Preferably, the substrate plates are constructed of a metal or alloy.

Any metal suitable for effect pigments can be used. Such metals include iron and steel, as well as all air- and water-resistant (semi)metals such as platinum, tin, zinc, chromium, molybdenum and silicon, as well as their alloys such as aluminum bronzes and brass. Preferred metals are aluminum, copper, silver and gold. Preferred substrate platelets include aluminum platelets and brass platelets, with aluminum substrate platelets being particularly preferred. Substrate plates made of aluminum can be produced, among other things, by punching out of aluminum foil or according to common milling and atomization techniques. For example, aluminum flakes are available from the Hall process, a wet milling process.

Other metal flakes, for example of bronze, can be obtained in a dry grinding process such as the Hametag process.

The substrate plates can have different shapes. For example, lamellar or lenticular metal platelets or so-called vacuum metallized pigments (VMP) can be used as substrate platelets. Lamellar substrate platelets are exemplified by an irregularly structured edge and are also referred to as “cornflakes” due to their appearance. Lenticular sustrate flakes have an essentially regular round edge and are also known as “silverdollars” because of their appearance.

The metal or metal alloy substrate plates can be passivated, for example by anodizing (oxide layer) or chromating.

A coating can change the surface properties and/or optical properties of the effect pigment and increase the mechanical and chemical load-bearing capacity of the effect pigments. For example, only the upper and/or lower side of the substrate wafer may be coated, with the side surfaces being recessed. Preferably, the entire surface of the optionally passivated substrate platelets, including the side surfaces, is covered by the layer. The substrate platelets are preferably completely encased by the coating.

The coating may include one or more layers. In a preferred embodiment, the coating has only layer A. In a likewise preferred embodiment, the coating has a total of at least two, preferably two or three, layers. It may be preferred to have the coating have two layers A and B, with layer B being different from layer A. Preferably, layer A is located between layer B and the surface of the substrate plate. In yet another preferred embodiment, the coating has three layers A, B and C. In this embodiment, layer A is located between layer B and the surface of the substrate wafer and layer C is located on top of layer B, which is different from the layer B below.

Suitable materials for the at least one layer, for example layers A and possibly B and C, are all substances that can be permanently applied to the substrate platelets. The materials should preferably be applicable in film form. Preferably, the entire surface of the optionally passivated substrate wafer, including the side surfaces, is enveloped by the at least one layer, for example, layer A or layers A and B or layers A, B and C.

The at least one layer is prepared wet-chemically using a metal alkoxide and an organosilicon compound having a basic group. The at least one layer comprises a metal oxide and/or metal oxide hydrate.

It is preferred that the metal oxide and/or metal oxide hydrate is selected from the group of silicon (di)oxide, silicon oxide hydrate, aluminum oxide, aluminum oxide hydrate, boron oxide, germanium oxide, manganese oxide, magnesium oxide, iron oxide, cobalt oxide, chromium oxide, titanium dioxide, vanadium oxide, zirconium oxide, tin oxide, zinc oxide and mixtures thereof.

Layer A preferably has at least one low refractive index metal oxide and/or metal oxide hydrate. Preferably, layer A comprises at least 95% by weight of low refractive index metal oxide (hydrate). Low refractive index materials have a refractive index of 1.8 or less, preferably 1.6 or less.

Low refractive index metal oxides suitable for Layer A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. Layer A preferably has a thickness of 1 to 100 nm, particularly preferably 5 to 50 nm, especially preferably 5 to 20 nm.

Layer B, if present, is different from Layer A and may contain at least one highly refractive metal oxide. Highly refractive materials have a refractive index of at least 1.9, preferably at least 2.0, and more preferably at least 2.4. Preferably, layer B comprises at least 95 wt. %, more preferably at least 99 wt. %, of high refractive index metal oxide(s).

If the layer B comprises a (highly refractive) metal oxide, it preferably has a thickness of at least 50 nm. Preferably, the thickness of layer B is no more than 400 nm, more preferably no more than 300 nm.

Highly refractive metal oxides suitable for layer B are, for example, selectively light-absorbing (i.e., colored) metal oxides, such as iron(III) oxide (α- and γ-Fe2O3, red), cobalt(II) oxide (blue), chromium(III) oxide (green), titanium(III) oxide (blue, usually present in admixture with titanium oxynitrides and titanium nitrides), and vanadium(V) oxide (orange), as well as mixtures thereof. Colorless high-index oxides such as titanium dioxide and/or zirconium oxide are also suitable.

Layer B can contain a selectively absorbing dye, preferably 0.001 to 5% by weight, particularly preferably 0.01 to 1% by weight, in each case based on the total amount of layer B. Suitable dyes are organic and inorganic dyes that can be stably incorporated into a metal oxide coating. Dyes in the sense of the present disclosure have a solubility in water (760 mmHg) at 25° C. of more than 0.5 g/L and are therefore not to be regarded as pigments.

Alternatively, to a metal oxide, layer B may comprise a metal particle carrier layer with metal particles deposited on the surface of the metal particle carrier layer. In a preferred embodiment, the metal particles directly cover a portion of the metal particle carrier layer. In this embodiment, the effect pigment has areas in which there are no metal particles, i.e., areas which are not covered with the metal particles.

The metal particle carrier layer comprises a metal layer and/or a metal oxide layer.

If the metal particle carrier layer comprises a metal layer and a metal oxide layer, the arrangement of these layers is not limited.

It is preferred that the metal particle support layer at least comprises a metal layer. It is further preferred that the metal layer comprises an element selected from tin (Sn), palladium (Pd), platinum (Pt) and gold (Au).

The metal layer can be formed, for example, by adding alkali to a metal salt solution comprising the metal.

If the metal particle carrier layer comprises a metal oxide layer, this preferably does not comprise silicon dioxide. The metal oxide layer preferably comprises an oxide of at least one element selected from the group of Mg (magnesium), Sn (tin), Zn (zinc), Co (cobalt), Ni (nickel), Fe (iron), Zr (zirconium), Ti (titanium) and Ce (cerium). Particularly preferably, the metal particle support layer iii) in the form of a metal oxide layer comprises a metal oxide of Sn, Zn, Ti and Ce.

The metal particle support layer in the form of a metal oxide layer can be produced, for example, by hydrolysis of an alkoxide of a metal forming the metal of the metal oxide in a sol-gel process.

The thickness of the metal particle support layer is preferably not more than 30 nm.

The metal particles may comprise at least one element selected from the group of aluminum (Al), titanium (Ti), chromium (Cr), iron (Fe), cobalt (Co), nickel (Ni), copper (Cu), zinc (Zn), ruthenium (Ru), rhodium (Rh), palladium (Pd), silver (Ag), tin (Sn), platinum (Pt), gold (Au), and alloys thereof. It is particularly preferred that the metal particles comprise at least one element selected from copper (Cu), nickel (Ni) and silver (Ag).

The average particle diameter of the metal particles is preferably not more than 50 nm, more preferably not more than 30 nm. The distance between the metal particles is preferably not more than 10 nm.

Suitable methods for forming the metal particles include vacuum evaporation, sputtering, chemical vapor deposition (CVD), electroless plating, or the like. Of these processes, electroless plating is particularly preferred.

According to a preferred embodiment, the effect pigments have a further layer C, comprising a metal oxide (hydrate), which is different from the layer B underneath. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The layer C preferably has a thickness of 10 to 500 nm, more preferably 50 to 300 nm.

The coating of the effect pigment has at least one layer that has been wet-chemically prepared from a metal alkoxide and an organosilicon compound having a basic group.

The at least one layer prepared using a metal alkoxide and an organosilicon compound having a basic group may be layer A, B and/or C. In the case where the coating has only layer A, layer A has been prepared using a metal alkoxide and an organosilicon compound having a basic group.

In the case where the coating of the effect pigment has two layers A and B, layer B has been prepared using a metal alkoxide and an organosilicon compound having a basic group.

In the case where the coating has layers A, B and C, layer C has been prepared using a metal alkoxide and an organosilicon compound having a basic group.

It is particularly preferred that the effect pigment has a substrate platelet of aluminum and a layer A comprising silica and the organosilicon compound having a basic group. Where the effect pigment based on a substrate platelet has a layer A and a layer C, it is preferred that the effect pigment has a substrate platelet of aluminum and layers A and C comprising silica, wherein the organosilicon compound having a basic group was further used to prepare the layer C.

It is essential to the present disclosure that an organosilicon compound having a basic group is used in the preparation of the at least one layer. The organic silicon compound is preferably a silane with one, two or three silicon atoms.

Particularly preferably, the organic silicon compound having a basic group further has one or more hydroxyl groups and/or hydrolysable groups per molecule.

These organic silicon compounds with a basic group are reactive compounds.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds of the present disclosure are compounds comprising one to three silicon atoms. Organic silicon compounds preferably contain one or two silicon atoms.

According to IUPAC rules, the term silane stands for a group of chemical compounds based on a silicone skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The basic group may preferably be an amino group, an alkylamino group, a dialkylamino group or a trialkylamino group, which is preferably connected to a silicon atom via a linker. Preferably, the basic group is an amino group, a C₁-C₆ alkylamino group or a di(C₁-C₆)alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably comprises a structural unit R′R″R″′Si—O—CH₂—CH₃. The radicals R′, R″ and R″′ represent the three remaining free valences of the silicon atom.

Preferred organic silicon compounds with a basic group have one or more basic groups and one or more hydroxyl groups or hydrolysable groups per molecule.

Particularly good results were obtained when the organic silicon compound with a basic group corresponds to formula (I) and/or (II).

The compounds of formulae (I) and (II) are organic silicon compounds selected from silanes having one, two or three silicon atoms, wherein the organic silicon compound comprises one or more hydroxyl groups and/or hydrolysable groups per molecule.

In another very particularly preferred embodiment, the organic silicon compound corresponds to formula (I) and/or (II),

R₁R₂N-L-Si(OR₃)ₐ(R₄)_{b} (I),

where
R₁, R₂ independently represent a hydrogen atom or a C₁-C₆ alkyl group,
L is a linear or branched divalent C₁-C₂₀ alkylene group,
R₃ is a hydrogen atom or a C₁-C₆ alkyl group,
R₄ represents a C₁-C₆ alkyl group
a, represents an integer from 1 to 3, and
b stands for the integer 3-a,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A, A′, A″, A″′ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. Very preferably, R₁ and R₂ both represent a hydrogen atom.

In the middle part of the organic silicon compound is the structural unit or the linker -L- which stands for a linear or branched, divalent C₁-C₂₀ alkylene group.

A divalent C₁-C₂₀ alkylene group may alternatively be referred to as a divalent or divalent C₁-C₂₀ alkylene group, by which is meant that each L grouping may form two bonds. One bond is from the amino group R1R2N to the linker L, and the second bond is between the linker L and the silicon atom.

Preferably, -L- represents a linear, divalent (i.e., divalent) C₁-C₂₀ alkylene group. Further preferably -L- stands for a linear divalent C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The linear propylene group (—CH₂—CH₂—CH₂—) can alternatively be referred to as the propane-1,3-diyl group.

The organic silicon compounds of formula (I)

R₁R₂N-L-Si(OR₃)ₐ(R₄)_{b} (I),

one end of each carries the silicon-comprising group —Si(OR₃)ₐ(R₄)_{b}.

In the terminal structural unit —Si(OR₃)ₐ(R₄)_{b}, R₃ is hydrogen or C₁-C₆ alkyl group, and R₄ is C₁-C₆ alkyl group. R₃ and R₄ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Particularly advantageous effect pigments could be produced if the organic silicon compound corresponds to formula (I), in which the radicals R₃, R₄ independently of one another stand for a methyl group or for an ethyl group.

Furthermore, advantageous effect pigments could be obtained if the organic silicon compound corresponds to formula (I), in which the radical a stands for the number 3. In this case the rest b stands for the number 0.

Particularly advantageous effect pigments could be obtained if the organic silicon compound corresponds to formula (I), wherein
R₃, R₄ independently of one another represent a methyl group or an ethyl group and
a stands for the number 3 and
b stands for the number 0.

Likewise, particularly advantageous effect pigments could be obtained if the organic silicon compound corresponds to formula (I),

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃ represents a hydrogen atom, an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

### (3-Aminopropyl)triethoxysilane

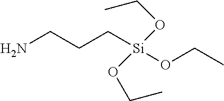

### (3-Aminopropyl)trimethoxysilane

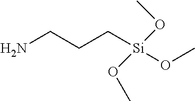

### 1-(3-Aminopropyl)silantriol

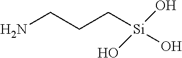

### (2-Aminoethyl)triethoxysilane

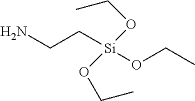

### (2-Aminoethyl)trimethoxysilane

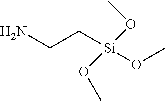

### 1-(2-Aminoethyl)silantriol

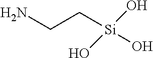

### (3-Dimethylaminopropyl)triethoxysilane

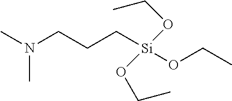 [SMILES: CCO[Si](CCCN(C)C)(OCC)OCC]

### (3-Dimethylaminopropyl)trimethoxysilane

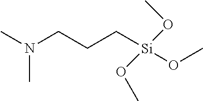

### 1-(3-Dimethylaminopropyl)silantriol

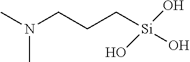

### (2-Dimethylaminoethyl)triethoxysilane

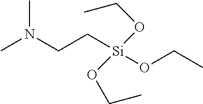

### (2-Dimethylaminoethyl)trimethoxysilane and

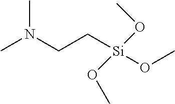

### 1-(2-Dimethylaminoethyl)silantriol

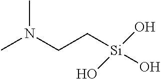

The organic silicon compounds of formula (I) are commercially available. (3-aminopropyl)trimethoxysilane, for example, can be purchased from Sigma-Aldrich. Also (3-aminopropyl)triethoxysilane is commercially available from Sigma-Aldrich.

Advantageous effect pigments could also be produced if the organic silicon compound corresponds to formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) each bear at their two ends the silicon-comprising groupings (R₅O)_{c}(R₆)_{d}Si— and —Si(R₆′)_{d′}(OR₅′)_{c′},

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein comprises at least one grouping from the group comprising -(A)- and —[NR₇-(A′)]- and —[O-(A″)]- and —[NR₈-(A″′)]-.

In the two terminal structural units (R₅O)_{c}(R₆)_{d}Si— and —Si(R₆′)_{d′}(OR₅′)_{c′}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

It is advantageous if the remainders c and c′ both stand for the number 3. In this case d and d′ both stand for the number 0.

Accordingly, advantageous effect pigments could be obtained if the organic silicon compound corresponds to formula (II)

(R₅O)_{c}(R₆)_{d}Si-(A)ₑ-[NR₇-(A′)]_{f}-[O-(A″)]_{g}-[NR₈-(A″′)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

When c and c′ both represent the number 3 and d and d′ both represent the number 0, the organic silicon compounds correspond to formula (IIa)

(R₅O)₃Si-(A)ₑ-[NR₇-(A′)]_{f}-[O-(A″)]_{g}-[NR₈-(A′″)]ₕ-Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are in the middle part of the organic silicon compound of formula (II).

In this context, the presence of certain groupings has proved to be particularly beneficial in terms of increasing washability. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A″′ and A″″ independently represent a linear or branched divalent C₁-C₂₀ alkylene group. Preferably the radicals A, A′, A″, A″′ and A″″ independently of one another represent a linear, divalent C₁-C₂₀ alkylene group. Further preferably the radicals A, A′, A″, A″′ and A″″ independently represent a linear divalent C₁-C₆ alkylene group. In particular, the radicals A, A′, A″, A″′ and A″″ independently of one another represent a methylene group (—CH₂—), an ethylene group (—CH₂—CH₂—), a propylene group (—CH₂—CH₂—CH₂—or a butylene group (—CH₂—CH₂—CH₂—CH₂—). Very preferably, the radicals A, A′, A″, A″′ and A″″ represent a propylene group (—CH₂—CH₂—CH₂—).

The divalent C₁-C₂₀ alkylene group may alternatively be referred to as a divalent or divalent C₁-C₂₀ alkylene group, by which is meant that each grouping A, A′, A″, A″′ and A″″ may form two bonds.

The linear propylene group (—CH₂—CH₂—CH₂—) can alternatively be referred to as the propane-1,3-diyl group.

If the radical f represents the number 1, then the organic silicon compound of formula (II) as contemplated herein comprises a structural grouping —[NR₇-(A′)]-.

If the radical f represents the number 1, then the organic silicon compound of formula (II) as contemplated herein comprises a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

When the radical f represents the number 1 and the radical h represents the number 0, the organic silicon compound as contemplated herein comprises the grouping [NR₇-(A′)] but not the grouping —[NR₈-(A′″)]. If the radical R7 now stands for a grouping of the formula (III), the agent (a) comprises an organic silicon compound with 3 reactive silane groups.

In a further preferred embodiment, an effect pigment is wherein the organic silicon compound corresponds to formula (II),

(R₅O)_{c}(R₆)_{d}Si-(A)ₑ-[NR₇-(A′)]_{f}-[O-(A″)]_{g}-[NR₈-(A″′)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group And
R7 represents a hydrogen atom, a methyl group, a 2-hydroxyethyl group, a 2-alkenyl group, a 2-aminoethyl group or a group of formula (III).

Organic silicon compounds of formula (II) which are well suited for solving the problem as contemplated herein are:

### 3-(Trimethoxysilyl)-N-[3-(trimethoxysilyl)propyl]-1-propanamine

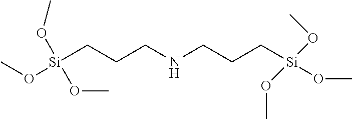

### 3-(Triethoxysilyl)-N-[3-(triethoxysilyl) propyl]-1-propanamine

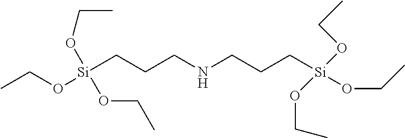 [SMILES: CCO[Si](CCCNCCC[Si](OCC)(OCC)OCC)(OCC)OCC]

### N-Methyl-3-(trimethoxysilyl)-N-[3-(trimethoxysilyl) propyl]-1-propanamine

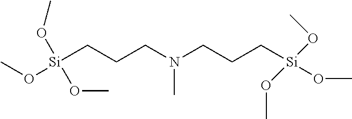

### N-Methyl-3-(triethoxysilyl)-N-[3-(triethoxysilyl) propyl]-1-propanamine

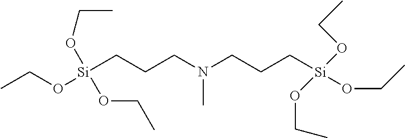 [SMILES: CCO[Si](CCCN(C)CCC[Si](OCC)(OCC)OCC)(OCC)OCC]

### 2-[Bis[3-(trimethoxysilyl) propyl]amino]-ethanol

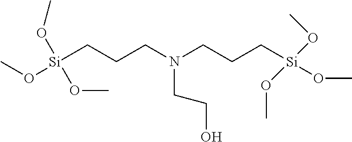

### 2-[Bis[3-(triethoxysilyl) propyl]amino]ethanol

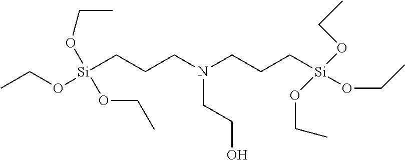 [SMILES: CCO[Si](CCCN(CCO)CCC[Si](OCC)(OCC)OCC)(OCC)OCC]

### 3-(Trimethoxysilyl)-N,N-bis[3-(trimethoxysilyl)propyl]-1-propanamine

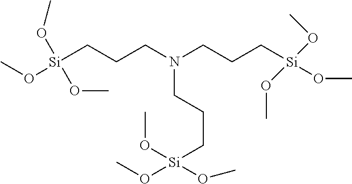

### 3-(Triethoxysilyl)-N,N-bis[3-(triethoxysilyl)propyl]-1-propanamine

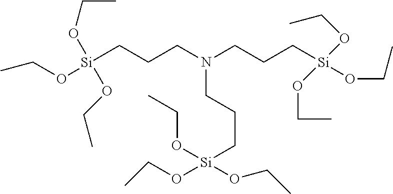 [SMILES: CCO[Si](CCCN(CCC[Si](OCC)(OCC)OCC)CCC[Si](OCC)(OCC)OCC)(OCC)OCC]

### N1,N1-Bis[3-(trimethoxysilyl)propyl]-1,2-ethanediamine,

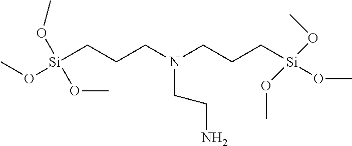

### N1,N1-Bis[3-(triethoxysilyl)propyl]-1,2-ethanediamine,

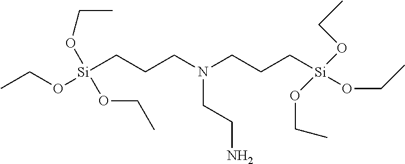 [SMILES: CCO[Si](CCCN(CCN)CCC[Si](OCC)(OCC)OCC)(OCC)OCC]

### N,N-Bis[3-(trimethoxysilyl)propyl]-2-propene-1-amine

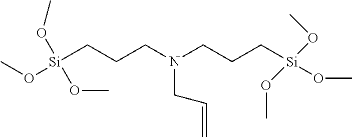

### N,N-Bis[3-(triethoxysilyl)propyl]-2-propene-1-amine

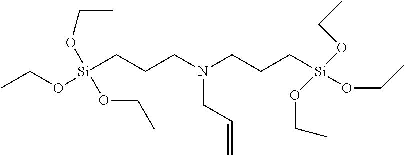 [SMILES: C=CCN(CCC[Si](OCC)(OCC)OCC)CCC[Si](OCC)(OCC)OCC]

The organic silicon compounds of formula (II) are commercially available. Bis(trimethoxysilylpropyl)amines with the CAS number 82985-35-1 can be purchased from Sigma-Aldrich.
Bis[3-(triethoxysilyl)propyl]amines with the CAS number 13497-18-2 can be purchased from Sigma-Aldrich, for example.
N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich or Fluorochem.
3-(triethoxysilyl)-N,N-bis[3-(triethoxysilyl)propyl]-1-propanamine with the CAS number 18784-74-2 can be purchased for example from Fluorochem or Sigma-Aldrich.

Even the addition of small amounts of water leads to hydrolysis in organic silicon compounds with basic groups and with at least one hydrolysable group. The hydrolysis products and/or organic silicon compounds with a basic group and with at least one hydroxy group and/or the hydrolysis products of the metal alkoxides can react with each other in a condensation reaction. For this reason, both the organosilicon compounds of basic group and with at least one hydrolysable group and their hydrolysis and/or condensation products as well as the condensation products with the hydrolysis products of the metal alkoxides can be included in the at least one layer. When organosilicon compounds with a basic group and with at least one hydroxyl group are used, both the organic silicon compounds with a basic group and with at least one hydroxyl group and their condensation products with themselves and/or with the hydrolysis products of the metal alkoxides can be included in the at least one layer.

A condensation product is understood to be either a product formed by the reaction of at least two organic silicon compounds each having at least one hydroxyl group or hydrolysable group per molecule with elimination of water and/or with elimination of an alkanol. The condensation products can, for example, be dimers, or even trimers or oligomers, where in the condensation products are always in balance with the monomers. Depending on the amount of water used or consumed in the hydrolysis, the equilibrium shifts from monomeric organic silicon compounds to condensation product.

A condensation product is also understood to mean a product that is formed by reacting at least one organic silicon compound of a basic group and having at least one hydroxyl group or hydrolysable groups per molecule with a hydrolysis product or condensation product of the metal alkoxides with elimination of water and/or with elimination of an alkanol.

By using acids and/or bases, the hydrolysis and/or the condensation reaction can be influenced. For example, the formation of the at least one layer can be influenced and controlled in terms of thickness, degree of condensation of the condensation products, degree of cross-linking of the condensation products, reaction rate.

The metal alkoxide used in the wet chemical coating process is preferably a silicon alkoxide selected from the group of tetramethyl orthosilicate, tetraethyl orthosilicate, tetraisopropyl orthosilicate and mixtures thereof, with tetraethyl orthosilicate being preferred.

Alternatively, aluminum alkoxides such as aluminum triisopropanolate or aluminum tri-sec-butanolate, zirconium alkoxides such as zirconium propylate, or titanium alkoxides such as titanium tetraethylate (tetraethyl orthotitanate) or titanium tetraisopropanolate (tetraisopropyl orthotitanate) can be used.

Layers A and C serve as corrosion protection as well as chemical and physical stabilization. Particularly preferably, layers A and C contain silicon dioxide or aluminum oxide applied by the sol-gel process.

The at least one layer may further comprise one or more colorant compounds selected from the group of pigments and/or direct dyes.

The particle size of the colorant compound used depends on the layer in which the colorant layer is present. The color-imparting compound preferably has a particle size D90, which is smaller than the layer thickness of the at least one layer. More preferably, the particle size D₉₅ of the coloring compound is smaller than the layer thickness of the at least one layer. Even more preferably, the particle size D₉₉ of the colorant compound is smaller than the layer thickness of the at least one layer. Very preferably, the particle size D₁₀₀ of the coloring compound is smaller than the layer thickness of the at least one layer. The particle size of the coloring compound can be determined using, for example, dynamic light scattering (DLS) or static light scattering (SLS). D₉₀ means that 90% of the particles of the coloring compound are smaller than the layer thickness of the at least one layer. Accordingly, D₉₅ means that 95% of the particles of the coloring compound are smaller than the layer thickness of the at least one layer, etc.

The amount of colorant compound selected from the group of pigments and/or direct dyes in the at least one layer is preferably up to 5% by weight, based on the total weight of the at least one layer.

Accordingly, it may be preferred that the at least one layer has been prepared wet-chemically using a metal alkoxide, an organosilicon compound having a basic group, and a coloring compound selected from the group of pigments and/or direct-drawing dyes.

Another subject matter of the application a process for preparing an effect pigment comprising a) a substrate platelet and b) a coating, comprising the steps:
(α) suspending the substrate wafer in an organic or aqueous solvent; and
(β) coating the substrate wafer suspended in step (α) by a sol-gel process using a metal alkoxide and an organosilicon compound having a basic group.

It is preferred that the metal alkoxide used in the sol-gel process is selected from the group of tetramethyl orthosilicate, tetraethyl orthosilicate, tetraisopropyl orthosilicate and mixtures thereof, with tetraethyl orthosilicate being preferred.

Alternatively, in addition to the tetraalkoxysilane, alkyltrialkoxysilanes may be used in the wet chemical coating process to produce the at least one layer, for example layer A or C.

Suitable alkyltrialkoxysilanes include, for example, methyltrimethoxysilane, methyltriethoxysilane, ethyltrimethoxysilane, ethyltriethoxysilane, propyltrimethoxysilane, propyltriethoxysilane, Hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and/or octadecyltriethoxysilane.

The organosilicon compound having a basic group is preferably selected from the group of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane
(2-Dimethylaminoethyl)trimethoxysilane
1-(2-Dimethylaminoethyl)silantriol and
Mixtures of these.

Very preferably, (3-dimethylaminopropyl)triethoxysilane and/or (3-dimethylaminopropyl)trimethoxysilane are used as organosilicon compounds with a basic group.

In a preferred embodiment of the manufacturing process, the substrate wafer used in step (a) has already been coated with at least one layer of a metal oxide and/or metal oxide hydrate.

An exemplary manufacturing process comprises dispersing the uncoated substrate platelets or the substrate platelets already coated with layer A or with layers A and B and the colorant compound selected from the group of pigments in a solution of a metal alkoxide such as tetraethyl orthosilicate or aluminum triisopropanolate (usually in a solution of organic solvent or a mixture of organic solvent and water with at least 50 wt. % organic solvent such as a C1 to C4 alcohol), and adding a weak base or acid to hydrolyze the metal alkoxide, thereby forming a film comprising the metal oxide and the colorant compound selected from the group of pigments on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-comprising layer to the oxide layers by annealing).

Although a mixture of two or more metal alkoxides can be used to produce the at least one layer, preferably layers A and/or C, only metal alkoxides of one metal, for example only silicon alkoxides or only aluminum alkoxides, are preferably used in each case to produce the at least one layer.

The effect pigments based on coated substrate platelets preferably have a thickness of 70 to 500 nm, particularly preferably 100 to 400 nm, especially preferably 150 to 320 nm, for example 180 to 290 nm. The low thickness of the coated substrate platelets is achieved by keeping the thickness of the uncoated substrate platelets low, but also by adjusting the thicknesses of the coatings A and, if present, C to as small a value as possible.

The adhesion and abrasion resistance of effect pigments based on substrate platelets to/in a material, preferably keratinous material, can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-comprising, layer A, B, or C. The outermost layer denotes the layer that is spatially farthest from the substrate platelet. The organic compounds are preferably functional silane compounds that can bind to the metal oxide-comprising layer A, B, or C. These can be either mono- or bifunctional compounds. Examples of bifunctional organic compounds are Methacryloxypropenyltrimethoxysilane, 3-Methacryloxypropyltrimethoxysilane, 3-Acryloxypropyltrimethoxysilane, 2-Acryoxyethyltrimethoxysilane, 3-Methacryoxypropyltriethoxysilane, 3-Acryloxypropyltrimethoxysilane, 2-Methacryloxyethyltriethoxysilane, 2-Acryloxyethyltriethoxysilane, 3-Methacryloxypropyltris(methoxyethoxy)silane, 3-Methacryloxypropyltris(butoxyethoxy)silane, 3-Methacryloxypropyltris(propoxy)silane, 3-Methacryloxypropyltris(butoxy)silane, 3-Acryloxypropyltris(methoxyethoxy)silane, 3-Acryloxypropyltris(butoxyethoxy)silane, 3-Acryloxypropyltris(butoxy)silane, Vinyltrimethoxysilane, Vinyltriethoxysilane, Vinylethyldichlorsilane, Vinylmethyldiacetoxysilane, Vinylmethyldichlorsilane, Vinylmethyldiethoxysilane, Vinyltriacetoxysilane, Vinyltrichlorsilane, Phenylvinyldiethoxysilane, or Phenylallyldichlorsilane. Furthermore, a modification with a monofunctional silane, an alkylsilane or arylsilane, can be carried out. This has only one functional group, which can covalently bond to the surface of the effect pigment (i.e., to the outermost metal oxide-comprising layer) or, if not completely covered, to the metal surface. The hydrocarbon residue of the silane points away from the effect pigment. Depending on the type and nature of the hydrocarbon residue of the same, a different degree of hydrophobicity of the effect pigment is achieved. Examples of such silanes are hexadecyltrimethoxysilane, propyltrimethoxysilane, etc. Particularly preferred are effect pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

### EXAMPLE

First, 200 g Al platelets in the form of VMPs (thickness between 20 nm and 30 nm, d₅₀=12 μm) were suspended in isopropanol. To this mixture 45 g of tetraethoxysilane and 2 g of (3-aminopropyl)trimethoxysilane were added and the resulting mixture was heated to 60° C. Subsequently, 100 g of water was added followed by 6 g of ammonia and the obtained mixture was stirred for another 4 h. The mixture is then filtered through a glass frit and the filter cake obtained is dried at 120° C. for 12 h. The filter cake is then removed. The layer obtained accounts for about 40 wt. %, based on the total weight of the effect pigment.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An effect pigment comprising:
a) a substrate platelet; and
b) a coating, wherein the coating comprises at least one layer which has been wet-chemically prepared using a metal alkoxide and an organosilicon compound having a basic group.

2. The effect pigment according to claim 1, wherein the coating completely envelops the substrate platelet.

3. The effect pigment according to claim 1, wherein the coating comprises a layer.

4. The effect pigment according to claim 1, wherein the coating has two or three layers.

5. The effect pigment according to claim 1, wherein the organic silicon compound is selected from silanes having one, two or three silicon atoms, and wherein the organic silicon compound comprises one or more hydroxyl groups and/or hydrolysable groups per molecule.

6. The effect pigment according to claim 1, wherein the basic group is selected from the group consisting of amino groups, alkylamino groups, dialkylamino groups, trialkylamino groups, and mixtures thereof.

7. The effect pigment according to claim 1, wherein the organosilicon compound with a basic group has the formula (I) and/or (II),
R₁R₂N-L-Si(OR₃)ₐ(R₄)_{b} (I),
where
R₁, R₂ independently represent a hydrogen atom or a C₁-C₆ alkyl group,
L is a linear or branched divalent C₁-C₂₀ alkylene group,
R₃ is a hydrogen atom or a C₁-C₆ alkyl group,
R₄ represents a C₁-C₆ alkyl group
a, represents an integer from 1 to 3, and
b stands for the integer 3-a,
(R₅O)_{c}(R₆)_{d}Si-(A)ₑ-[NR₇-(A′)]_{f}-[O-(A″)]_{g}-[NR8-(A′″)]ₕ-Si(R₆′)_{d′}(OR₅′)_{c′} (II),
where
R5, R5′, R5″ independently represent a hydrogen atom or a C₁-C₆ alkyl group,
R6, R6′ and R6″ independently represent a C₁-C₆ alkyl group,
A, A′, A″, A″′ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
where
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0.

8. The effect pigment according to claim 1, wherein the at least one layer has been applied using a sol-gel process.

9. The effect pigment according to claim 1, further comprising a monofunctional or bifunctional organic compound bound to the further coating.

10. The effect pigment according to claim 1, wherein the substrate platelet is of a metal, preferably aluminum, or of an alloy.

11. A process of making an effect pigment comprising a substrate platelet and a coating, comprising the steps:
suspending the substrate platelet in an organic or aqueous solvent; and
coating the substrate platelet by a sol-gel process using a metal alkoxide and an organosilicon compound having a basic group.

12. The process of claim 11, wherein the metal alkoxide is selected from the group consisting of tetramethyl orthosilicate, tetraethyl orthosilicate, tetraisopropyl orthosilicate, and mixtures thereof.

13. The process of claim 11, wherein the organosilicon compound is selected from the group consisting of:
(3-Aminopropyl)trimethoxysilane;
(3-Aminopropyl)trimethoxysilane;
1-(3-Aminopropyl)silantriol;
(2-Aminoethyl)triethoxysilane;
(2-Aminoethyl)trimethoxysilane;
1-(2-Aminoethyl)silantriol;
(3-Dimethylaminopropyl)triethoxysilane;
(3-Dimethylaminopropyl)trimethoxysilane;
1-(3-Dimethylaminopropyl)silantriol;
(2-Dimethylaminoethyl)trimethoxysilane;
(2-Dimethylaminoethyl)trimethoxysilane;
1-(2-Dimethylaminoethyl)silantriol; and
mixtures thereof.

14. The process according to claim 11, wherein an alkyltrialkoxysilane is further used in the sol-gel process.

15. The process according to claim 11, wherein the substrate platelet is coated with at least one layer of a metal oxide and/or metal oxide hydrate before coating the substrate platelet by a sol-gel process using a metal alkoxide and an organosilicon compound having a basic group.

16. The process of claim 11, wherein the metal alkoxide is tetraisopropyl orthosilicate.
